(19) United States
(12) Patent Application Publication
(10) Pub. No.: US 2019/0175900 A1
(43) Pub. Date: Jun. 13, 2019

(54) LOW FREQUENCY ELECTROTHERAPY DEVICE

(71) Applicant: CROWN MEDICAL CO., LTD., Seoul (KR)

(72) Inventors: HYEJEONG LEE, GYEONGGI-DO (KR); HUNIL CHO, SEOUL (KR); JEONGHUN PARK, SEOUL (KR); JAEMIN HAN, SEOUL (KR); GYEONGMIN KIM, SEOUL (KR); JUNG SOON CHO, SEOUL (KR); SANG HO MOON, SEOUL (KR)

(21) Appl. No.: 16/186,896

(22) Filed: Nov. 12, 2018

(30) Foreign Application Priority Data
Dec. 7, 2017 (KR) 10-2017-0167597

(51) Int. Cl. A61N 1/04; A61N 1/32

(52) U.S. Cl. CPC A61N 1/0456; A61N 1/0484; A61N 1/08; A61N 1/0428; A61N 1/321

(57) ABSTRACT

The present invention relates to a low frequency electrotherapy device, which can transfer poles into a human body as they are without causing a short circuit between a positive pole (+) and a negative pole (−) in order to correct ion imbalance of a user's affected part and provide help to treat insomnia without side effects, can be used widely to reduce a treatment period of time since being easily detachably attached to the user's proper position and expanding a treatment range using both poles or multiple poles, and can maximize treatment effects since being used while adjusting the number of poles according to conditions of the user's affected part. The low frequency electrotherapy device includes: a power source unit for supplying a predetermined DC power to circuit units by rectifying commercial electricity through a bridge; a CPU for controlling the entire operation; a display unit for displaying previously set operations; a control unit for inputting a control signal; a buzzer unit for generating sounds whenever the device starts and ends operation and the control signal is inputted; an output control unit for controlling strength of output; a pulse generating unit for inputting a predetermined oscillation frequency signal by regulating pulse generation frequencies; and an output unit having rectifier circuits isolated from transformers individually mounted at output terminals, wherein a plurality of output terminals, which output independent monopoles to prevent a short circuit between a positive pole and a negative pole to generate low frequencies, and the pulse generating unit includes pulse generating terminals, and a plurality of the pulse generating terminals and a plurality of the output units are connected with each other in parallel to simultaneously select a plurality of electrodes. The output unit is located at one side of a band body and the band body is fastened by a fastening means in such a way that an output terminal of the output unit comes into contact with a user's shoulder or neck, so that the output unit outputs poles to the user's shoulder and/or neck for a predetermined period of time.

### BACKGROUND OF THE INVENTION

### Field of the Invention

The present invention relates to a low frequency electrotherapy device, and more particularly, to a low frequency electrotherapy device, which can transfer poles into a human body as they are without causing a short circuit between a positive pole (+) and a negative pole (−) in order to correct ion imbalance of a user's affected part and provide help to treat insomnia without side effects, can be used widely to reduce a treatment period of time since being easily detachably attached to the user's proper position and expanding a treatment range using both poles or multiple poles, and can maximize treatment effects since being used while adjusting the number of poles according to conditions of the user's affected part.

### Background Art

Low frequency is a sort of electric wavelengths, and has an electrical property capable of obtaining physical therapy effect by stimulating a human body's transcutaneous nerves using frequency with a band of 3 to 2000 Hz.

The human body has about 60 trillion cells, and all of the cells generate bioelectricity, and it has been reported that low frequency therapy devices are very effective to activate weak bioelectricity.

An affected part of the human body has positive ions and negative ions of an incomplete state generated by metabolite, and in this instance, the positive ions are increased by uniting among the positive ions and the negative ions are increase by uniting among the negative ions. It interrupts a normal blood flow and causes extravasated blood and congestion with blood, and prevents a smooth supply of nutrient materials and oxygen to press capillaries and nerve tissues. Such affected parts may be a habitat of germs to cause various diseases. If the positive ions and the negative ions are excessively accumulated inside and outside cell membranes, a potential difference and tensity are increased to cause diseases.

The ions of the incomplete state are united among the ions with the same polarity because they cannot obtain objects that they demand, namely, electrons and quantums. The reason is caused by the physicochemical action that the poles are increased by uniting among poles with the same polarity and push among poles with the same polarity after obtaining their relative polarity since the poles are incomplete before obtaining their relative polarity.

Therefore, electrons and quantums generated from the electrotherapy device adjust balance among ions by promoting union, neutralization and dissolution among the ions of the incomplete state at the affected part through a give-and-take action of dual characteristics.

When the ions of the incomplete state are balanced, dissolution of inflammatory products at the affected part is promoted, and cells and tissues are regenerated normally. Moreover, during the union and neutralization among elements generated from the therapy device and the ions of the incomplete state, momentary heat of neutralization is generated, and in this instance, a sterilization action is caused. Therefore, the therapy device can recover a control function and an immune function of the human body, and eradicate and prevent diseases.

A conventional medical electrotherapy device has used DC, AC, PC for physical treatment (high frequency, medium frequency, and low frequency). Because the conventional electrotherapy device outputs a positive (+) pole and a negative pole (−) from the same circuit, they have friendly relative relationship, so cause a short circuit by using the human body as a conductor. Therefore, because the conventional therapy device does not have the give-and-take action with the human body's affected part and the human body serves just as a conductor for electricity, disease causing materials are not removed. Therefore, the conventional therapy device gives a temporary physical effect by heat, vibration and stimulus, but may be harmful to human bodies.

In the meantime, in order to solve the above problems, recently, a single pole therapy device has been disclosed. However, the single pole therapy device has a disadvantage in that it causes imbalance of potential in a human body's tissues and cells since inducing a unilateral change in the human body by using the single pole among both poles of electricity.

Therefore, the conventional electrotherapy devices are difficult to treat diseases since they cannot correct imbalance of ions and potential difference which are causes of diseases. Additionally, the conventional electrotherapy devices are difficult to treat diseases since some of people avoid electric therapy devices due to side effects by instant electric shock and electrical burn of the tissues by a short circuit.

In order to solve the above problems, Korean Patent Application No. 2002-8257 discloses a non-short circuit bipolar electrotherapy device. However, in fact, the bipolar electrotherapy device shows a short circuit of a small amount, so people demands an electrotherapy device capable of allowing users to use in safety.

Furthermore, it is necessary to reduce the size of the bipolar electrotherapy device to be portable.

### PATENT LITERATURE

### Patent Literatures

Patent Document 1: Korean Patent Application No. 2002-8257 entitled “Non-short circuit bipolar electrotherapy device”

### SUMMARY OF THE INVENTION

Accordingly, the present invention has been made to solve the above-mentioned problems occurring in the prior arts, and it is an object of the present invention to provide a low frequency electrotherapy device, which can transfer poles into a human body as they are without causing a short circuit between a positive pole (+) and a negative pole (−) in order to correct ion imbalance of a user's affected part and provide help to treat insomnia without side effects, can be used widely to reduce a treatment period of time since being easily detachably attached to the user's proper position and expanding a treatment range using both poles or multiple poles, and can maximize treatment effects since being used while adjusting the number of poles according to conditions of the user's affected part.

To accomplish the above object, according to the present invention, there is provided a low frequency electrotherapy device including: a power source unit for supplying a predetermined DC power to circuit units by rectifying commercial electricity through a bridge; a CPU for controlling the entire operation; a display unit for displaying previously set operations; a control unit for inputting a control signal; a buzzer unit for generating sounds whenever the device starts and ends operation and the control signal is inputted; an output control unit for controlling strength of output; a pulse generating unit for inputting a predetermined oscillation frequency signal by regulating pulse generation frequencies; and an output unit having rectifier circuits isolated from transformers individually mounted at output terminals, wherein a plurality of output terminals, which output independent monopoles to prevent a short circuit between a positive pole and a negative pole to generate low frequencies, and the pulse generating unit includes pulse generating terminals, and a plurality of the pulse generating terminals and a plurality of the output units are connected with each other in parallel to simultaneously select a plurality of electrodes. The output unit is located at one side of a band body and the band body is fastened by a fastening means in such a way that an output terminal of the output unit comes into contact with a user's shoulder or neck, so that the output unit outputs poles to the user's shoulder and/or neck for a predetermined period of time.

Moreover, the fastening means is a Velcro tape.

Furthermore, the band body is a band with a predetermined length, and both end portions of the band body are detachably combined by the fastening means.

Additionally, the band body includes: a waist belt part fixed on the user's waist by the fastening means; and an X band part formed on the waist belt part in the shape of alphabet X, wherein the output unit is located at one side of the X band part to be slung over the user's shoulder.

Moreover, the rectifier circuit is a full-wave rectifier circuit or half-wave rectifier circuits.

In addition, the output terminal protrudes outwardly from the inner face of the band body.

According to the present invention, the low frequency electrotherapy device can transfer poles into a human body as they are without causing a short circuit between a positive pole (+) and a negative pole (−) in order to correct ion imbalance of a user's affected part and provide help to treat insomnia without side effects, can be used widely to reduce a treatment period of time since being easily detachably attached to the user's proper position and expanding a treatment range using both poles or multiple poles, and can maximize treatment effects since being used while adjusting the number of poles according to conditions of the user's affected part.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIGS. 1A and 1B are views showing a low frequency electrotherapy device according to the present invention;

FIGS. 2A and 2B are views showing a used state of FIGS. 1A and 1B;

FIG. 3 is a view showing a brief configuration of the low frequency electrotherapy device according to the present invention;

FIG. 4 is a view showing a pulse generating unit and an output unit according to the present invention; and

FIG. 5 is a view showing the output unit having a rectifier circuit according to the present invention.

### DETAILED DESCRIPTION OF THE PREFERRED EMBODIMENT

Hereinafter, an embodiment of the present invention will be described in detail with reference to the accompanying drawings. However, the embodiments are exemplary for describing the present disclosure more specifically, and it will be understood by those skilled in the art that the scope of the present disclosure is not limited by the embodiments.

Now, according to preferred embodiments, technical structure of the present invention will be described in detail as follows.

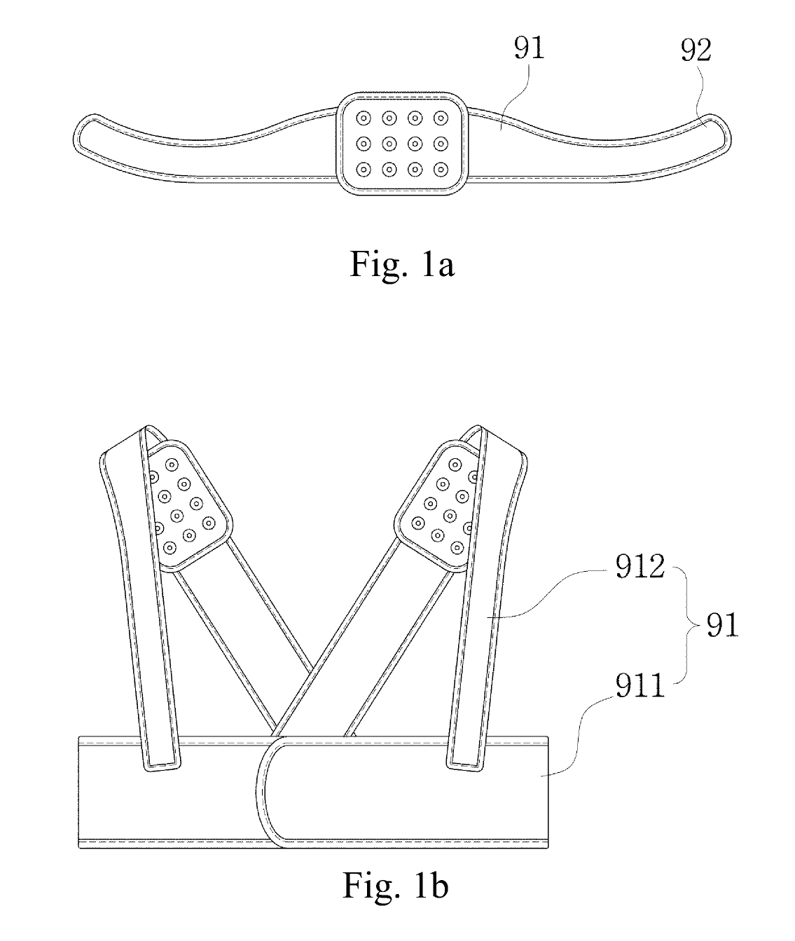

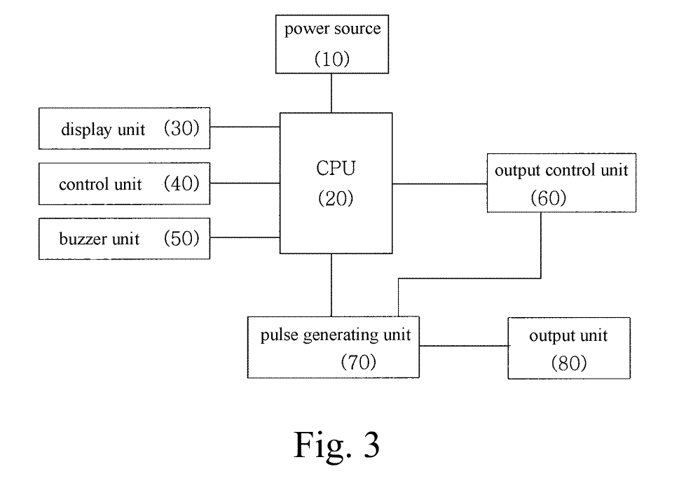

FIG. 1 is a view showing a low frequency electrotherapy device according to the present invention, FIG.>2 is a view showing a used state of FIG. 1, and FIG. 3 is a view showing a brief configuration of the low frequency electrotherapy device according to the present invention.

As shown in FIGS. 1 to 3, the low frequency electrotherapy device according to the present invention includes: a power source unit 10 for supplying a predetermined DC power to circuit units by rectifying commercial electricity through a bridge; a CPU 20 for controlling the entire operation; a display unit 30 for displaying previously set operations; a control unit 40 for inputting a control signal; a buzzer unit 50 for generating sounds whenever the device starts and ends operation and the control signal is inputted; an output control unit 60 for controlling strength of output; a pulse generating unit 70 for inputting a predetermined oscillation frequency signal by regulating pulse generation frequencies; and an output unit 80 having rectifier circuits 81 and 82 isolated from transformers T1 and T2 individually mounted at output terminals, wherein a plurality of output terminals, which output independent monopoles to prevent a short circuit between a positive pole and a negative pole to generate low frequencies. The pulse generating unit 70 includes pulse generating terminals 71 and 72, and a plurality of the pulse generating terminals 71 and 72 and a plurality of the output units 80 are connected with each other in parallel to simultaneously select a plurality of electrodes. The output unit 80 is located at one side of a band body 91 and the band body 91 is fastened by a fastening means 92 in such a way that an output terminal of the output unit 80 comes into contact with a user's shoulder or neck, so that the output unit 80 outputs poles to the user's shoulder and/or neck for a predetermined period of time.

Especially, in case of biofeedback therapy for patients who suffer from chronic insomnia, the band body 91 is fastened by the fastening means 92 in such a way that the output terminal of the output unit 80 comes into contact with the user's shoulder or neck so as to act to the user's shoulder or neck, which is the highest in muscle tone. Therefore, the low frequency electrotherapy device comes into close contact with the user's shoulder and/or neck at the output unit 80, and the output unit 80, which has a plurality of the output terminals for outputting independent monopoles to prevent a short circuit between the positive pole and the negative pole, generates low frequencies. That is, a plurality of the output terminals transfer stimulation to the user's shoulder or neck.

In this instance, the fastening means 92 is detachably mounted using one among well-known various means, such as Velcro tapes, buttons, and others. As shown in FIG. 1A, the band body 91 is a band with a predetermined length, and both end portions of the band body 91 are detachably combined by the fastening means 92 so that the user can easily wear the low frequency electrotherapy device on the user's neck.

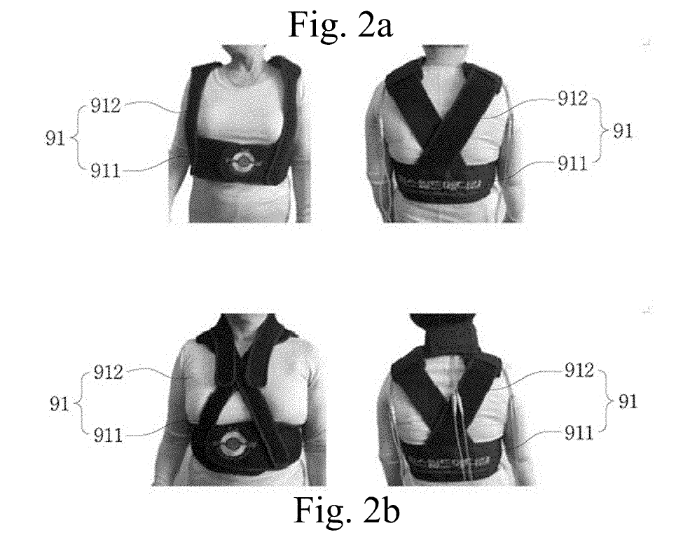

Moreover, as shown in FIGS. 1B, 2A and 2B, the band body 91 includes a waist belt part 911 fixed on the user's waist by the fastening means 92; and an X band part 912 formed on the waist belt part 911 in the shape of alphabet X, wherein the output unit 80 is located at one side of the X band part 912 to be slung over the user's shoulder. When the user slings the X band part 912 over the shoulder and carries the waist belt part 911 on the waist to easily fix the low frequency electrotherapy device in stability, the low frequency electrotherapy device can directly give a stimulus on the user's shoulder.

Especially, the output terminals of the output units 80 protrude outwardly from an inner face of the band body 91 to directly give stimulus to the user's affected part. Therefore, the low frequency electrotherapy device can stimulate the user's affected part and pressurize the affected part by the output terminals of the protrusion shape to provide a massage effect.

That is, as shown in the drawings, a plurality of the output terminals are disposed to be spaced apart from each other at regular intervals and protrude outwardly in the protrusion type so as to enhance therapy effect by directly pressurizing and stimulating the user's affected part.

FIG. 3 is a brief view of the low frequency electrotherapy device including the power source unit 10, the CPU 20, the display unit 30, the control unit 40, the buzzer unit 50, the output control unit 60, the pulse generating unit 70, and the output unit 80 having a plurality of the independent full-wave rectifier circuits or half-wave rectifier circuits 81 and 82.

The power source unit 10 rectifies commercial AC power source to supply a predetermined AC power source to the circuit units.

The display unit 30 displays setting of operations of the low frequency electrotherapy device according to the present invention.

The control unit 40 receives a control signal when the user sets the operation of the low frequency electrotherapy device.

The buzzer unit 50 generates sounds when the user inputs the control signal and when the low frequency electrotherapy device starts to operate.

The pulse generating unit 70 generates a predetermined oscillation frequency signal, outputs the signal to the output unit 80, and a plurality of the pulse generating units 70 are individually disposed at the output terminals of the output units 80.

Each of the output units 80 includes the transformers T1 and T2 and the full-wave rectifier circuits or half-wave rectifier circuits 81 and 82 at the output terminals to form independent output circuits.

The output control unit 60 controls output inputted to the output unit 80.

The CPU 20 controls operations of the above-mentioned components.

The output units 80 and the pulse generating units 70 are connected with one another in parallel to output a lot of poles.

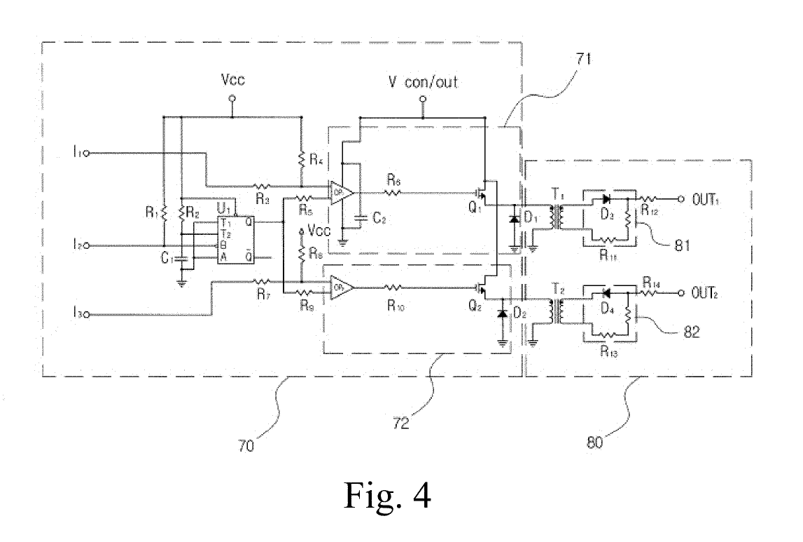

FIG. 4 is a view showing a pulse generating unit and an output unit according to the present invention.

As shown in FIG. 4, the pulse generating unit 70 includes: input terminals I1, I2 and I3 receiving a frequency generation signal through the CPU 20; amplifier circuits OP1 and OP2 for amplifying the frequency generation signal to a predetermined level; and transistor circuits Q1 and Q2 for generating pulses. The output unit 80 includes: transformers T1 and T2 for receiving the pulse signal outputted from pulse generation terminals 71 and 72 of the pulse generating unit 70 to convert the pulse signal into AC power; half-wave rectifier circuits 81 and 82 for changing + and − AC signals, which come from the transformers, into a signal having just one polarity; and output terminals OUT1 and OUT2 for outputting the signal to be applied to a human body.

In detail, Vcc is the base voltage inputted from the power source unit 10, and Vcon/out is an output control voltage inputted from the output control unit 60 to the pulse generating unit 70.

When a frequency generation signal to actuate the CPU is inputted to the input terminals I1, I2 and I3, the frequency generating signal is amplified to a predetermined level through the amplifier circuits OP1 and OP2 of the pulse generating terminals 71 and 72. In order to accurately operate the signal, a trigger circuit U1 which is triggered with a predetermined width is connected to the amplifier circuits OP1 and OP2. Here, R1, R2, R3, R4, R7, R8 and R9 are resistances for adjusting strength of input voltage, and C1 is a capacitor disposed on the trigger circuit U1.

A capacitor C2 connected to the amplifier circuit OP1 modulates signals inputted to the transistor Q1 and the transistor Q2 to be different from each other, and R6 and R10 reduce the size of signals inputted to the transistors Q1 and Q2 not to exceed an input range of the transistors Q1 and Q2.

D1 and D2 are disposed to protect circuits when overload is applied, and R12 and R14 are disposed to increase and decrease output voltage.

Now, operation of the low frequency electrotherapy device according to the present invention will be described as follows.

First, when a frequency generating signal to operate the CPU 20 is inputted to the input terminals I1, I2 and I3, the frequency generating signal is amplified to the predetermined level through the amplifier circuits OP1 and OP2 of the pulse generating terminals 71 and 72, and then, inputted to the transistors Q1 and Q2. If potential of the signal is more than the minimum potential for operating the transistors Q1 and Q2, the transistors Q1 and Q2 are turned on. However, if the potential of the signal is less than the minimum potential, the transistors Q1 and Q2 are turned off. Pulses are generated by the ON-OFF actions of the transistors Q1 and Q2, and strength of the generated pulses is adjusted according to strength of Vcon/out.

The generated pulses are inputted to the transformers T1 and T2 to be converted into an AC signal, and are operated as independent circuits which do not have any influence on each other.

The transformer T1 has the half-wave rectifier circuit 81, which operates in case of a + value and outputs a + half-wave AC signal to the output terminal OUT1, and the transformer T2 has the half-wave rectifier circuit 82, which operates in case of a − value and outputs a − half-wave AC signal to the output terminal OUT2.

Therefore, the output unit 80 has the half-wave rectifier circuits 81 and 82 respectively disposed at the transformers T1 and T2 in order to output monopoles.

The monopoles are independently formed not to cause a short circuit between a positive pole and a negative pole, and are transferred into a human body as they are in order to correct ion imbalance of the user's affected part and to regenerate cellular tissues.

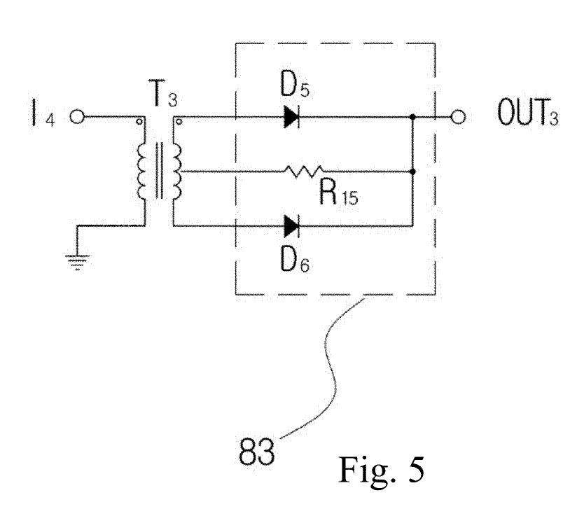

FIG. 5 is a view showing the output unit 80 having the full-wave rectifier circuit 83 according to the present invention. Output voltage is adjusted by the full-wave rectifier circuit 83, and is applicable to the electrotherapy device according to the present invention.

When the input terminal 14 receives signals outputted from the pulse generating terminals 71 and 72 of the pulse generating unit 70, a + full-wave rectifier signal is outputted to the output terminal OUT3 through diodes D5 and D6 and a resistance R15 after passing through a transformer T3.

When input and output directions of the diodes are changed simply, since a − full-wave rectifier circuit may be realized, description of the − full-wave rectifier circuit will be omitted.

In this embodiment, it is described that the output unit 80 includes the half-wave rectifier circuits 81 and 82 and outputs positive poles and negative poles. However, the output unit 80 may include a full-wave rectifier circuit according to use purposes and may output both poles of (+, +) or (−, −). Alternatively, the output unit 80 may output multiple poles (+, −+, −+, . . . ) by connecting a plurality of the output terminals, which include transformers and rectifier circuits, in parallel.

In order to fine the effect of curing insomnia by the low frequency electrotherapy device according to the present invention, the following experiment has been carried out.

### Experimental Example

Age: 55 years or more
The number of persons: 55 persons
Period: 28 days
Object: Insomnia patients who suffer from insomnia for more than six months and have been treated with medicine
How to use: The patients used the low frequency electrotherapy device according to the present invention during a predetermined period of time from half an hour before the time to take insomnia-related medicine for more than five days a week, and in case of biofeedback for the insomnia patients, the low frequency electrotherapy device was applied to the patient's shoulders and neck, which are the highest in muscle tone.

Average use time a day was 39.7±9.3 minutes. Average current intensity was 137.1±20.3 μA at the neck and 138.8±20.9 μA at the shoulders. Low frequency electrostimulation was greatly improved at PSQI (from 12.53±3.65 to 11.05±3.73, Cohen's d=0.403, p<0.001) and at ISI sore (from 13.48±7.24 to 11.72±5.98, Cohen's d=0.265, p=0.006).

Among total 55 persons, six persons withdrawn their consent due to inconvenience in use of the device or troubles on the skin, two persons were worsened in insomnia, one person showed hypoglycemia, one person felt fatigue, and one person died. So, the 11 persons quit the experiment, and effectiveness analysis index on 44 persons was measured.

For sleep diaries and questionnaire survey, the subjects completed their sleep diaries the next morning after their night electrostimulation session. The check list in the diary includes sleep latency, the period of time that the subjects were in bed, delayed sleep, working strength and period of TENS, and dosage.

Effectiveness analysis index of Pittsburgh sleep quality index (PSQI), Epworth sleepiness scale (ESS), and insomnia severity index (ISI) was measured, and vitals signs including blood pressure and pulse were also checked. Because the patients' moods or pain conditions may have influences on insomnia symptoms, hospital anxiety and depression scale (HADS) and numeral rating scale (NRS) were also measured, and the measurement result was shown in the following Table 1.

TABLE 1

| | Pre-treatment | Post-treatment | P-value |
|---|---|---|---|
| PSQI | 12.42 ± 3.73 | 11.0 ± 3.71 | 0.001** |
| Sleep latency | 56.00 ± 43.03 | 37.77 ± 23.49 | 0.001** |
| Time in bed | 408.60 ± 78.64 | 430.12 ± 67.40 | 0.033** |
| total sleep time | 311.28 ± 87.00 | 334.42 ± 85.92 | 0.004** |
| sleep efficiency | 77.17 ± 18.59 | 78.6 ± 19.07 | 0.506 |
| ISI | 13.88 ± 7.23 | 12.10 ± 6.08 | 0.004** |
| ESS | 3.74 ± 3.79 | 4.10 ± 3.33 | 0.408 |
| Sleep Diary | | | |
| sleep quality | 2.33 ± 1.24 | 2.38 ± 1.32 | 0.728 |
| daytime function | 1.55 ± 1.04 | 1.55 ± 1.11 | 1.000 |
| QOL | 1.38 ± 1.41 | 1.19 ± 1.17 | 0.160 |
| HADA | 13.81 ± 8.15 | 13.72 ± 7.78 | 0.881 |
| NRS | 2.72 ± 2.38 | 2.88 ± 2.37 | 0.534 |

As shown in the Table 1, the 44 patients showed the results that PSQI (from 12.42±3.73 to 11.0±3.71, P=0.001) and ISI (from 13.88±7.23 to 12.10±6.08, P=0.004) were reduced after the experiment.

Among the 44 patients, six patients reduced benzodiazepine dosage after treatment, 23 patients showed therapy reaction (Response rate of 57.5%). In comparison of changes in sleep variables written in the sleep diaries with respect to a treated group and an untreated group, sleep latency time was reduced (from 44.46±26.9 minutes to 30.26±17.55 minutes, p<0.001, Cohen's d=0.625), and sleep period was increased (from 308.7±82.2 minutes to 347±77.66 minutes, p=0.001, Cohen's d=0.479). Treatment was predicted by an increase of the base line of ESS score and an increase of the base line of HADS score. The two scores were all obtained after age, gender, other mental diseases, period of insomnia, and quantity and intensity of treatment were adjusted. Relative delta electric power in the occipital region was reduced in the treated group (from 16.5±9.7 to 10.9±8.9) rather than the untreated group (from 13.7±9.1 to 14.5±11.9).

After the low frequency electrotherapy, the sleep latency time and waking time were reduced. Furthermore, through polysomnography which may contribute to treatment of insomnia, it was found that slow wave-form sleep and total delta sleep were increased considerably. Differently from a positive role of delta during the sleep period, delta waves increased in an awareness condition showed a lot of pathological states, such as brain tissue damage and a decline in cognitive functions. Delta waves during brain awake time were generally caused by fatigue and mental boredom due to insomnia, and showed a meaningful decrease of relative delta power in the awareness condition. It is related with decrease in subjective degree of insomnia, and a difference in decrease of relative delta power between the treated group and the untreated group proved that delta power decreased in the awareness condition showed treatment effect by use of low frequency electrostimulation.

Positive results were found from low frequency electrostimulation carried out to trapezius muscles at 1 mA or less during a session of 30 minutes to 60 minutes for more than 20 days (Average 137 μA to 138 μA). Selection of a portion to be stimulated is as important as the selection of proper period and level of electrostimulation. Especially, because anatomical and functional innervation of the cervical nerve in the trapezius muscle was checked, high muscle stress of the trapezius muscle is generally observed when insomnia patients get muscle relaxation treatment in a sleeping clinic since psychological anxiety on sleep disorder is a strong predictive factor of an increased muscle reaction of the upper trapezius muscle. Therefore, it is preferable to carry out low frequency electrostimulation on the patient's neck or shoulders.

Mechanism actions for relieving insomnia through low frequency electrostimulation are as follows. First, the low frequency electrostimulation is effective to neuromuscles. It is shown that persons who suffer from physiological problems, such as sleep disorder experience muscle contraction when a surface electromyogram is recorded bidirectionally in the trapezius region and the deltoid region. Because positive relations between muscle relaxation and improvement of sleep quality is clear, the low frequency electrostimulation on muscles showed that expansion of muscles was increased and spasticity of cerebral palsy patents was decreased. Additionally, blood supply, skin temperature, and muscle oxidative capacity were improved, and inflammatory cytokine was restrained. Second, neurophysiological effects of the brain may be induced by the low frequency electrostimulation.

In comparison of states before and after the low frequency electrostimulation treatment, meaningful improvements in PSQI and ISI were observed, and it showed therapeutic effect of insomnia. More than half of the insomnia patients showed that the low frequency electrostimulation using the low frequency electrotherapy device according to the present invention was effective in decrease of serious insomnia without side effects. Moreover, the electrostimulation helped some of the patients reduce use of sleeping pills, and was more effective to patients who suffer from high daytime sleepiness and high depressive anxiety.

Therefore, the low frequency electrotherapy device can transfer poles into a human body as they are without causing a short circuit between a positive pole (+) and a negative pole (−) in order to correct ion imbalance of a user's affected part and provide help to treat insomnia without side effects, can be used widely to reduce a treatment period of time since being easily detachably attached to the user's proper position and expanding a treatment range using both poles or multiple poles, and can maximize treatment effects since being used while adjusting the number of poles according to conditions of the user's affected part. Therefore, the low frequency electrotherapy device can transfer poles into a human body as they are without causing a short circuit between a positive pole (+) and a negative pole (−) in order to correct ion imbalance of a user's affected part and provide help to treat insomnia without side effects, can be used widely to reduce a treatment period of time since being easily detachably attached to the user's proper position and expanding a treatment range using both poles or multiple poles, and can maximize treatment effects since being used while adjusting the number of poles according to conditions of the user's affected part.

As described above, while the present invention has been particularly shown and described with reference to the example embodiments thereof, it will be understood by those of ordinary skill in the art that various changes, modifications and equivalents may be made in the present invention without departing from the technical scope and idea of the present invention. Therefore, it would be understood that the present invention is not limited by the changes, modifications and equivalents but is limited by the following claims.

What is claimed is:

1. A low frequency electrotherapy device comprising:
a power source unit for supplying a predetermined DC power to circuit units by rectifying commercial electricity through a bridge;
a CPU for controlling the entire operation;
a display unit for displaying previously set operations;
a control unit for inputting a control signal;
a buzzer unit for generating sounds whenever the device starts and ends operation and the control signal is inputted;
an output control unit for controlling strength of output;
a pulse generating unit for inputting a predetermined oscillation frequency signal by regulating pulse generation frequencies; and
an output unit having rectifier circuits isolated from transformers individually mounted at output terminals, wherein a plurality of output terminals, which output independent monopoles to prevent a short circuit between a positive pole and a negative pole to generate low frequencies, and the pulse generating unit includes pulse generating terminals, and a plurality of the pulse generating terminals and a plurality of the output units are connected with each other in parallel to simultaneously select a plurality of electrodes,
wherein the output unit is located at one side of a band body and the band body is fastened by a fastening means in such a way that an output terminal of the output unit comes into contact with a user's shoulder or neck, so that the output unit outputs poles to the user's shoulder and/or neck for a predetermined period of time.

2. The low frequency electrotherapy device according to claim 1, wherein the fastening means is a Velcro tape.

3. The low frequency electrotherapy device according to claim 1, wherein the band body is a band with a predetermined length, and both end portions of the band body are detachably combined by the fastening means.

4. The low frequency electrotherapy device according to claim 1, wherein the band body includes:
a waist belt part fixed on the user's waist by the fastening means; and
an X band part formed on the waist belt part in the shape of alphabet X, wherein the output unit is located at one side of the X band part to be slung over the user's shoulder.

5. The low frequency electrotherapy device according to claim 1, wherein the rectifier circuit is a full-wave rectifier circuit or half-wave rectifier circuits.

6. The low frequency electrotherapy device according to claim 1, wherein the output terminal protrudes outwardly from the inner face of the band body.
